(19) United States
(12) Patent Application Publication
(10) Pub. No.: US 2011/0184413 A1
(43) Pub. Date: Jul. 28, 2011

(54) Ankle Fusion Plate

(76) Inventor: Gordon Slater, Albury (AU)

(21) Appl. No.: 12/300,890

(22) PCT Filed: May 17, 2007
(86) PCT No.: PCT/AU2007/000656

§ 371 Date: Dec. 17, 2010

(30) Foreign Application Priority Data
May 17, 2006 (AU) 2006902652
May 17, 2007 (AU) PCT/AU2007/000656

(51) Int. Cl. A61B 17/80

(52) U.S. Cl. 606/70

(57) ABSTRACT

A fusion plate for arthrodesis, the plate comprising: a plate body including a first portion disposed in a first plane and having a first bone engaging surface and a second opposing surface, a second portion disposed in a second plane and having a first bone engaging surface and a second opposing surface, a third portion disposed in a third plane and having a first bone engaging surface and a second opposing surface. The first portion includes at least one opening which receives at least one fixation means. The second portion includes at least one opening which receives at least one fixation means and the third portion includes at least one opening which receives at least one fixation screw.

### BACKGROUND

The present invention relates to prosthetic devices and more particularly relates to an ankle fusion plate for fusion of the anterior ankle. More particularly the invention relates to an ankle plate in which openings in the plate receive fixation screws allowing compression of bones being fused. The invention further, relates to a method of insertion of an anterior ankle plate so that optimal compression is achieved in anterior ankle joint fusion. This invention further relates to a kit including a selection of ankle fusion plates and a selection of fasteners for fixation of the ankle plate in a prescribed manner such that orientation of the screws provide optimal compression and therefore mechanical advantage. More particularly, this invention relates to an improved apparatus for fusion of ankle joints.

### PRIOR ART

The prior art is replete with orthopaedic devices for repairing bones and particularly diseased bones and bone fractures. The prior art teaches a variety of bone fixation systems using plates and screws.

For example, U.S. Pat. No. 6,235,034 discloses a bone plate comprising a base plate having at least two screw holes and at least two bone screws capable of securing the bone plate to a bone by insertion through the screw holes into the bone. The bone screws have heads shaped to toggle within the screw holes. A retaining plate is provided that is fixedly attachable to the base plate. The retaining plate covers at least a portion of each of the bone screws. The retaining plate and base plate each contain set screw apertures. A set screw is provided to retain the retaining plate in place over the base plate by screwing the set screw through the set screw apertures in the retaining plate and base plate. This design prevents the bone screw from backing out from the bone once screwed in through the base plate.

Another example of plate fixation by screw is disclosed in U.S. Pat. No. 5,951,558 which discloses a fixation device for, keeping two or more bone pieces together, either pieces of a broken bone or two distinct bones, for undergoing junction of the pieces by natural welding. The device described is capable of immobilizing flat or round bones, long or short bones, for example, parts of a broken femur or two contiguous vertebrae. The device comprises a fixation plate and fixation screws, the plate having orifices for passing the screws through the plate and fastening the screws into the bone tissue. A screw blocking or locking mechanism is provided in the plate to block the screws in the fastening position once the screws have been passed through the plate and screwed into the bone pieces, for preventing the screws from unscrewing from the bone pieces and moving out of the fixation plate once and after the fixation plate and the screws have been firmly installed in the bones.

Another prior art bone fusion device is taught in U.S. Pat. No. 6,830,589 which describes expandable bone fusion devices and methods of use. The fusion device according to the invention described in that patent includes a first member and a second member which can be deployed and locked into an expanded configuration to stabilize the adjacent bone during fusion of the bone.

More generally U.S. Pat. No. 6,663,669 discloses a total ankle replacement system and novel surgical method for total ankle replacement. Novel surgical tools for performing the surgical method are also described. The total ankle replacement system includes the calcaneus in fixation of a lower prosthesis body, thereby significantly increasing the amount of bone available for fixation of the lower prosthesis body and allowing the lower prosthesis body to be anchored with screws. The total ankle replacement system further includes a long tibial stem which can also be anchored into the tibia with, for example, screws, nails, anchors, or some other means of attachment.

Bones which have been fractured, either by accident or severed by surgical procedure, must be kept together, for lengthy periods of, time in order to permit the recalcification and bonding of the severed parts. Accordingly, adjoining parts of a severed or fractured bone are typically clamped together or attached to one another by means of plates, pins or screws driven through the rejoined parts. Movement of the pertinent part of the body may then be kept at a minimum, such as by application of a cast, brace, splint, or other conventional technique, in order to promote healing and avoid mechanical stresses that may cause the bone parts to separate during bodily activity. The surgical procedure of attaching two or more parts of a bone with a pin-like device normally requires an incision into the tissue surrounding the bone and the drilling of a hole through the bone parts to be joined. Due to the significant variation in bone size, configuration, and load requirements, a wide variety of bone fixation devices have been developed in the prior art. In general, the current methods rely upon a variety of metal wires, screws, plates and clamps to stabilize the bone fragments during the healing process. Following a sufficient bone healing period of time, the site may require re-opening to permit removal of the bone fixation device.

The internal fixation techniques commonly followed today frequently rely upon the use of wires, intramedullary pins, plates and screws, and combinations thereof. The particular device or combination of devices is selected to achieve the best anatomic and functional condition of the traumatized bone with the simplest operative procedure and with a minimal use of foreign-implanted stabilizing material. It is important in bone repair that the fracture be stable axially, torsionally and rotationally.

### Ankle Fusion

The goal of ankle replacement is to resurface the ankle joint with mechanical parts that allow continued ankle motion and function without pain. The models currently used include the Agility Ankle and the Scandinavian Total Ankle Replacement (STAR). Not all known systems are approved for use. Like hip and knee replacements, these devices are constructed of meals and plastics, and, as such, are mechanical parts that can wear out. Currently, patients who are best served by an ankle replacement are those who will put low mechanical demands on their artificial joints. These include average or lightweight patients who would like to stand and walk with limited or no pain. Certain patient groups are less likely to have good and long-lasting results from ankle replacements, including patients with; previous deep ankle infection, lower limb neuropathy, osteoporosis, high physical demands, obesity, poor skin or vascular problems. Ankle arthroplasty for post-traumatic tibiotalar arthritis remains controversial. The current literature strongly recommends arthrodesis, especially in those patients who will overload the joint: the young, the active and overweight patients. Total ankle arthroplasty has become a viable alternative to ankle arthrodesis. Selected patients can be offered a total ankle replacement as an alternative option to arthrodesis in the treatment of end-stage ankle arthritis. The optimal total ankle replacement patient is an older person who applies low loads and has multiple joint problems.

The ankle joint is a comparatively small joint relative to the weight bearing and torque it must withstand. Total ankle replacement systems attempting to address pain control and improved function have in the past experienced significant failure rates due to the technical difficulty of simulating ankle geometry and loadings. The main alternative to total ankle replacement is arthrodesis. Both procedures are intended to reduce pain but the total ankle replacement is additionally intended to improve function. If an arthrodesis or ankle replacement is not properly aligned, significant gait abnormalities may result. The principal limitations of past total ankle replacement have been loosening of the prosthesis, requiring revision. If the prosthesis requires removal, a subsequent arthrodesis can be considered. Different prostheses require different amounts of removal of bone, potentially compromising the success of a subsequent arthrodesis. Some ankles with implant components often require revision or arthrodesis. Ankle arthrodesis is currently a widely accepted surgical procedure but good, uniform results are not always achievable. Patients treated by compression ankle arthrodesis do not always have an effective fusion rate. The commonly used external fixation devices afford stability in only one plane and do not give rigid immobilization. A device known as a Triangular Compression Device has been used successfully. A successful Arthrodesis of the ankle can result in a painless, normal walking gait. However, complications in ankle arthrodesis can be major, and can occur when anatomy, deformity, or bony deficiency is not properly addressed. Arthrodesis is usually considered after conservative treatment (such as arthroscopy) fails. Infections, deformity, sensory deficiencies, and bony defects are complications which require special consideration. External compression enhances the likelihood of a successful arthrodesis.

Presently ankle fusion has had a rate of failure in the literature between 15% and 70%. It is believed that this is largely a result of Initial fixation that is not rigid enough. As in other area of the skeletal body, superior fusion rates have been achieved by plating rather than fixation by other means Pantalar fusion has been achieved with a revision foot rod. This has been a very technically difficult device to use with poor fusion rates.

Attempts have in the past been made in the prior art to bend and shape an already existing plate on the market but this does not fulfil the requirements, as screws cannot be placed in the desired places or angles to achieve optimal fixation.

A known plate marketed under the trade mark name Tomofix™ has been crudely adapted for anterior arthrodesis of the ankle but this is unsatisfactory because the plate is designed for stable fixation of osteotomies close to the knee.

Proper plate fixation relies on the integrity of the screw bone interface, screw insertion angle, screw tightness and effective co operation between screw head and the screw insertion hole. These requirements dictate plate design for a particular anatomical, location and repair objective. To date there is no arthrodesis plate and particularly ankle arthrodesis plate which satisfies the requisite fixation criteria and which is purpose designed to overcome the prior art disadvantages of the known plates.

Since the ankle is the only joint which to date does not have a specific plate for arthrodesis, there is a long felt want in the field to provide a fusion plate that is effective and useful in primary ankle fusion and which will reduce or eliminate fusion failure rates and which provides appropriate geometry to facilitate integrity of the screw bone interface, screw insertion angle, screw tightness and effective co operation between screw head and the screw insertion hole.

### INVENTION

The present invention seeks to ameliorate or eliminate the aforesaid problems inherent in the prior art devices and apparatuses and particularly those used in ankle arthrodesis.

The present invention provides an improved arthrodesis fusion plate for fusion of the anterior ankle. More particularly the invention provides an ankle plate in which openings in the plate receive fixation screws allowing compression of bones being fused and orientation of the fixation screws to optimise accommodation of bone loading for efficient and effective fusion. The invention further provides a method of insertion of an anterior ankle plate so that optimal compression is achieved and fixation screws are inserted at appropriate angles in anterior ankle joint fusion. This invention further relates to a kit including an anterior ankle fusion plate and which includes a selection of fasteners for fixation of the ankle plate in a prescribed manner so that the orientation of the screws provide optimal, compression and bone fusion.

This ankle fusion plate according to the invention can be inserted into the anterior ankle joint and is used in primary ankle fusion, covetin severe hind foot deformity, pantalar fusion and also in the salvage of ankle replacement.

Although the invention will be described with reference to its application to ankle fusion it will be appreciated by persons skilled in the art that the invention may be applied to the repair/fusion of other bones requiring axial alignment.

In one broad form the present invention comprises:

a fusion plate for arthrodesis, the plate comprising:
a plate body including a first portion disposed in a first plane and having a first bone engaging surface and a second opposing surface,
a second portion disposed in a second plane and having a first bone engaging surface and a second opposing surface,
a third portion disposed in a third plane and having a first bone engaging surface and a second opposing surface,
the first portion including at least one opening which receives at least one fixation means;
the second portion including at least one opening which receives at least one fixation means;
the third portion including at least one opening which receives at least one fixation screw.

In another broad form the present invention comprises:
an implant kit for arthrodesis fusion the kit comprising:
a fusion plate; and
a set of plate fixation screws;
the plate comprising, a first portion disposed in a first plane and having inner and outer surfaces, the inner surface opposing a bone surface, the first portion including at least one opening including a formation which receives at least one bone fixation screw in a first orientation, a second portion disposed in a second plane and having inner and outer surfaces, the inner surface opposing a hone surface, the second portion including at least one opening including a formation which receives a bone fixation screw in at least one orientation, a third portion disposed in a third plane and having inner and outer surfaces, the inner surface opposing a bone surface, the third portion including at least one opening which receives at least one bone fixation screw in a first orientation.

In its broadest form the present invention comprises:
a fusion plate for arthrodesis, the plate comprising:
a plate body including a first portion disposed in a first plane and having a first bone engaging surface and a second opposite surface,
a second portion disposed in a second plane and having a first bone engaging surface and a second opposing surface,
the first portion including at least one opening which receives at least one fixation means;
the second portion including at least one opening which receives at least one fixation means.

Preferably the inner surface of the first portion of the plate opposes the anterior tibia. Preferably the inner surface of the second portion of the plate also opposes the anterior tibia. Preferably, the inner surface of the third portion of the plate disposed in the first plane opposes the talus. The first portion includes at least one opening including a formation which receives a plurality of bone screws of said first type and which on insertion of the plate are disposed normal to the plane of the plate at that region. The second portion of the plate includes a slotted opening which receives a screw of a second, type which is of sufficient length to penetrate the tibia, talus and Calcaneus bones. The third portion preferably has two spaced apart openings which receive at least one of a first screw type which are implanted into the Talus.

According to one embodiment, the first portion has a region at is extremity which is thinner.

The screws in each portion of the plate are directed at required angles according to the joint/s required for, arthrodesis. This is also necessary to achieve maximal compression of the fusion site/s. The fixation screw design is adapted to ensure the above plate fitting objectives are achieved.

According to a preferred embodiment, the plate depth changes at different locations. Preferably, the depth at the beginning arid end points of the L shaped contour over the ankle joint in the second region will be at its maximum thickness. This location adjacent the ankle joint will preferably be the thickest part of the plate and will preferably fall within the range 4-8 mm. Thickness throughout this specification will refer to the dimension measured from the bone engaging face to an opposite outer face The plate will taper at at least one but preferably two different points of the plate. A first taper will occur at a proximal point of the plate over the tibia. The desired effect is for the plate to taper in and decrease in thickness proximally. The taper decreases down to around 1 mm in thickness and at its proximal extent it is 4 mm in width. The second point of plate depth and width change is over the phalanges at the distal point of the plate. At this point the plate according to one embodiment, capers out and again the thickness at this point would be in the order of about 1 min. These points will preferably resemble and conform to the typical geometry of the anatomical region. In locations where this does not occur, further manipulation or moulding of the plate geometry can be achieved as required. Preferably, the plates are configured to generally conform to the anatomic contours of the ankle joint. A range of different plate sizes (at least five) is contemplated with differences in the range of contour and degree of angle over the ankle joint and lengths both proximally and distally. In practice 2-3 three plate sizes are likely to provided a complete inventory.

### BRIEF DESCRIPTION OF THE DRAWINGS

The present invention will now be described in more detail according to a preferred, but non limiting embodiment and with reference to the accompanying illustrations wherein:

FIG. 1 shows a side elevation view of a plate according to one embodiment and attached via fixation screws to an abbreviated ankle joint (dotted lines)

FIG. 2 shows a front elevation view of, the plate of FIG. 1 showing alignment and spacing of predrilled holes for fixation screws.

FIG. 3 shows an elevation view of, a first screw type according to one embodiment adapted for insertion in openings for the tibia.

FIG. 4 shows an elevation view of a second screw type according to one embodiment adapted for insertion in the plate of FIGS. 1 and 2 and allowing adjustable orientation.

FIG. 5 shows a side cross sectional elevation view of a plate according to a preferred embodiment isolated from an ankle joint.

FIG. 6 shows a front elevation view of the plate of, FIG. 5 with corresponding numbering.

FIG. 7 shows a perspective view of the plate of FIG. 5 with corresponding numbering.

FIG. 8 shows a cross sectional view of the plate of FIG. 5 taken at A-A in FIG. 6.

FIG. 9 shows the cross sectional side elevation view of the plate of FIG. 5 (taken along line A of FIG. 10) showing a non limiting geometry of the plate according to one embodiment.

FIG. 10 shows a front elevation view of the plate of FIG. 9 showing a non limiting geometry of the plate according to one embodiment.

FIG. 11 shows a cross sectional view of the plate of FIG. 10 taken at D-D in FIG. 10 showing a non limiting geometry of the plate according to one embodiment.

FIG. 12 shows an enlarged view of detail B in FIG. 9 showing a non limiting geometry of the opening.

FIG. 13 shows an enlarged view of detail C in FIG. 9 showing a non limiting geometry of the opening.

### DETAILED DESCRIPTION OF THE PREFERRED EMBODIMENT

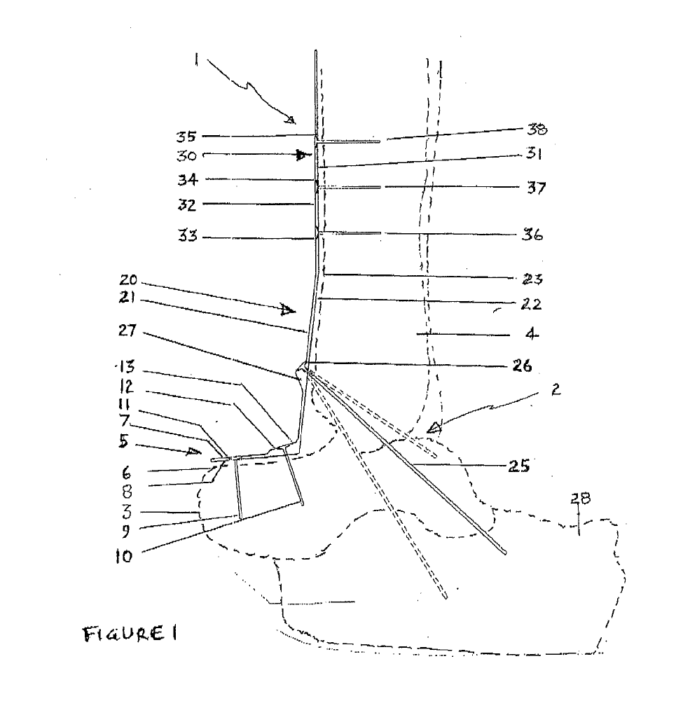

Referring to FIG. 1 there is shown a side elevation generally schematic view of a fusion plate 1 for arthrodesis according to one embodiment. Plate 1 is attached to an ankle joint 2 opposing the Talus bone 3 and Tibial bone 4.

Plate 1 according to the embodiment shown comprises a portion 5 disposed in a first plane which generally aligns with an anterior surface 6 of, the talus 3. Portion 5 has an outer surface 7 and inner surface 8 which opposes talus surface 6 for, fixation thereto. Disposed in portion 5 are fixation screws 9 and 10 which pass through openings 11 and 12 of portion 5 and engage talus 3 at different orientations. Screws 9 and 10 are disposed at different angles to a vertical resulting in each having different respective horizontal and vertical components of force along orthogonal X and Y axes. Each of openings 11 and 12 have formations which direct respective screws 9 and 10 in the orientations shown. For example the angle of orientation of countersink in formation 13 of opening 12 directs screw 10 at a predetermined angle which optimises fixation.

Portion 20 of plate 1 has an outer surface 21 and inner surface 22 which opposes anterior surface 23 of tibia 4 for fixation thereto. Disposed in portion 20 is fixation screw 25 which passes through opening 26 in formation 27. Formation 27 is configured so that screw 25 is implanted at an angle within a predetermined allowable angular range. The allowable range will preferably be within a 40 degree arc. Screw 25 engages tibia 4, talus 3, and calcaneus 28 effectively providing three points of fixation according to this embodiment. Portion 20 is angled relative to portion 5 at about 100 degrees. This angle is non limiting and may be greater or less than 100 degrees according to the dictates of design.

Portion 30 of plate 1 has an inner surface 31 and an outer surface 32 and preferably disposed normal or near normal to the plane of portion 5. Portion 30 includes openings 33, 34 and 35 which receive fastening screws 36, 37 and 38 each preferably in the same orientation and which engage tibia 4. Screws 36, 37 and 38 are according to one embodiment 4.5 mm in diameter and may be of the same screw type as those used for screws 9 and 10 fixing portion 5. This diameters is non limiting. Also a different typo of fixation screw to the configuration of that shows may be employed.

As may be seen from FIG. 1, the screws are placed in a particular orientation and required angle to the joint/s required for arthrodesis. This is also necessary to achieve maximal compression of the fusion site/s.

Preferably, portion 30 is disposed in a first plane which generally aligns with an opposing face of tibia 4. Portion 20 lies in a second plane at a first angle relative to the first plane and aligns with an opposing face of the distal tibia. Portion 5 lies in a third plane at a second angle relative to the first plane and engages the talus.

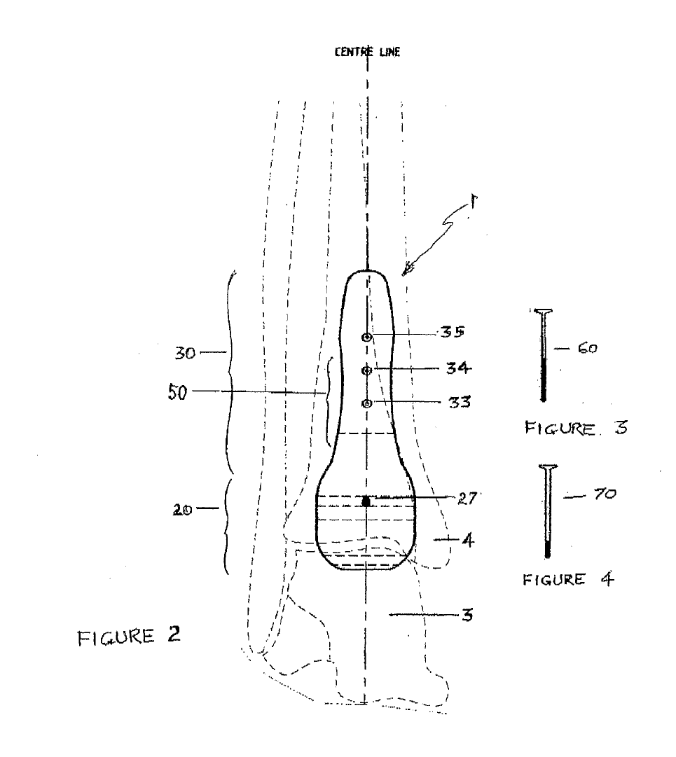

FIG. 2 shows a front elevation, view of the plate of FIG. 1 showing alignment and spacing of predrilled holes for fixation screws. FIG. 2 has corresponding numbering for corresponding parts. From this view it may be seen that according to this embodiment, plate 1 undergoes changes along its length depending upon the bone each part of the plate opposes. Region 30 is narrower than region 20 tapering as the tibia narrows heading proximally. In addition, the loads at region 30 can be absorbed by the thinner profile as internal moments are less. As the plate moves distally, the thickness preferably increases to accommodate the increased compression and rotational loads. The waisted region 50 generally confirms to the contour of the tibia. Openings 33, 34 and 35 are preformed and receive a first preferably countersunk screw type such as that shown in FIG. 3. Opening 27 which is also preformed, receives a countersink screw which is allowed adjustable orientation. The enlarged slotted holes allow a fine adjustment of the attitude of the screws within a range of around 30 degrees, although it will be appreciated that this range can be narrowed or extended.

FIG. 3 shows an elevation view of a first screw type 60 according to one embodiment adapted for insertion in openings 33, 35 and 36 for the tibial region fixation.

FIG. 4 shows an elevation view of a second screw type 70 according to one embodiment adapted for insertion in the plate of FIGS. 1 and 2 and allowing adjustable orientation. Screw type 70 has a longer shank to increase depth of penetration and has an abbreviated threaded portion to allow the majority of the shank to slide through aligned tibial and talus screw holes finally anchoring in the calcaneus bone.

FIG. 5 shows a side elevation view of a plate 80 according to a preferred embodiment isolated from an ankle joint. Plate 80 which is attachable to an ankle joint opposing the Talus bone and Tibial bone, comprises a portion 81 disposed in a first plane which generally aligns with an anterior surface of the talus. Portion 81 has an outer surface 82 and inner surface 83 which opposes a talus for fixation thereto. Disposed in portion 81 are fixation screws (not shown) which pass through openings 84 and 85 of portion 81 engaging the talus at selected orientations. Screws inserted in openings 84 and 85 are preferably disposed at different angles to a vertical resulting in each having different respective horizontal and vertical components of force along orthogonal X and Y axes. Each of openings 84 and 85 have formations 86 and 87 which direct screws in a particular orientation. The angle of orientation of countersink formations 86 and 87 directs screws at a predetermined angle which optimises fixation.

Portion 90 of plate 80 has an outer surface 91 and inner surface 92 which opposes an anterior surface of tibia for fixation thereto. Disposed in portion 90 is a fixation screw which passes through opening 93 in formation 94. Formation 94 is configured so that a fixation screw is directed at an angle within a predetermined allowable angular range. Portion 90 is angled relative to portion 81 at a non limiting angle of about 100 degrees.

Portion 95 of plate 1 has an inner surface 96 and an outer surface 97 and preferably disposed normal or near normal to the plane of portion. Portion 95 includes openings 98 and 99 which receive fastening screws each preferably in the same orientation and which engage the tibia. Screws which fix plate 80 via openings 98 and 99 may be of the same screw type as those used for fixation through openings 84 and 85.

As may be seen from FIG. 1 the screws are placed in a particular orientation and required angle to the joints required for arthrodesis. This is also necessary to achieve maximal compression of the fusion site/s.

Preferably, portion 95 is disposed in a first plane which generally aligns with an opposing face of a tibia. Portion 90 lies in a second plane at a first angle relative to the first plane and also aligns with an opposing face of, the distal tibia. Portion 5 lies in a third plane at a second angle relative to the first plane and engages the talus. Openings 99 and 100 are elongated to allow alignment adjustments.

FIG. 6 shows a front elevation view of the plate of FIG. 5 with corresponding numbering.

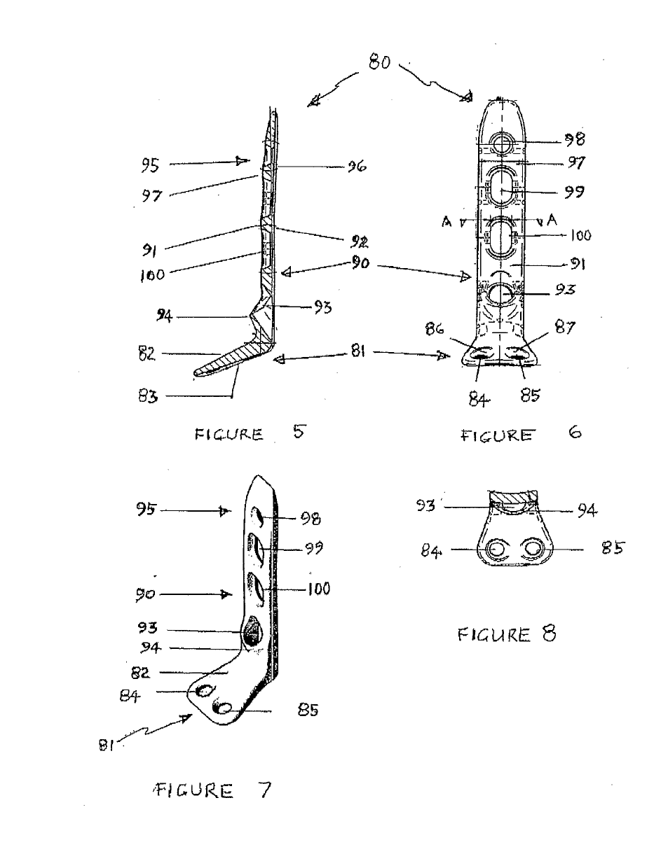

FIG. 7 shows a perspective view of the plate of FIG. 5 with corresponding numbering.

FIG. 8 shows a cross sectional and plan views of the plate of FIG. 5 taken at A-A in FIG. 6.

According to a preferred embodiment the depth/thickness of the plate 80 will change at different locations on the plate. The depth at the beginning and end points of the generally L shaped contour over the ankle joint formed by portions 81 and 90 will be at it's maximum thickness as it is at this region that the highest loading will occur in normal use. This depth (the thickest part of the plate) will be within the range 5-6 mm. The plate will taper at two different points on the plate. Firstly, the proximal end region of the plate over the tibia. The desired effect is for the plate to taper in and decrease depth at its extremities where loading is least. This would decrease down to around 1 mm in thickness and 4 mm in width. The second point of plate depth and width change would be over the phalanges at the distal point of the plate. At this point the plate would taper out and again the depth would be that of around the 1 mm mark. These points will resemble the average geometry of this anatomical region in the cases where this is not occurring it is at these points that further manipulation or moulding can be achieved if required.

In view of the anatomic contour of ankle joint, there are at least five different sized plates with differences in the range of contour and degree of angle over the ankle joint and lengths both proximally and distally. Typically a kit selection or, inventory would provide choice of two or three plate sizes.

Preferably, the custom shaped L-plate would need to be moulded to an angle in the region of 110 degrees at the point of the contact with the patient bone surface. The range in sizes for this would preferably be 95, 100, 105 110 and 115 degrees, with corresponding lengths to suit.

An ideal tibial length of the plate would be approximately 80 mm. The contour of geometry over the distal aspect of the tibia will be incorporated into the design of the plate. The length from the L point out to the phalanges would be about 25 mm in length and at this point the plate would taper out to around 35 mm. The plate width before tapering in or out would be in the region of 11 min. One preferred material for the plate is chrome Cobalt.

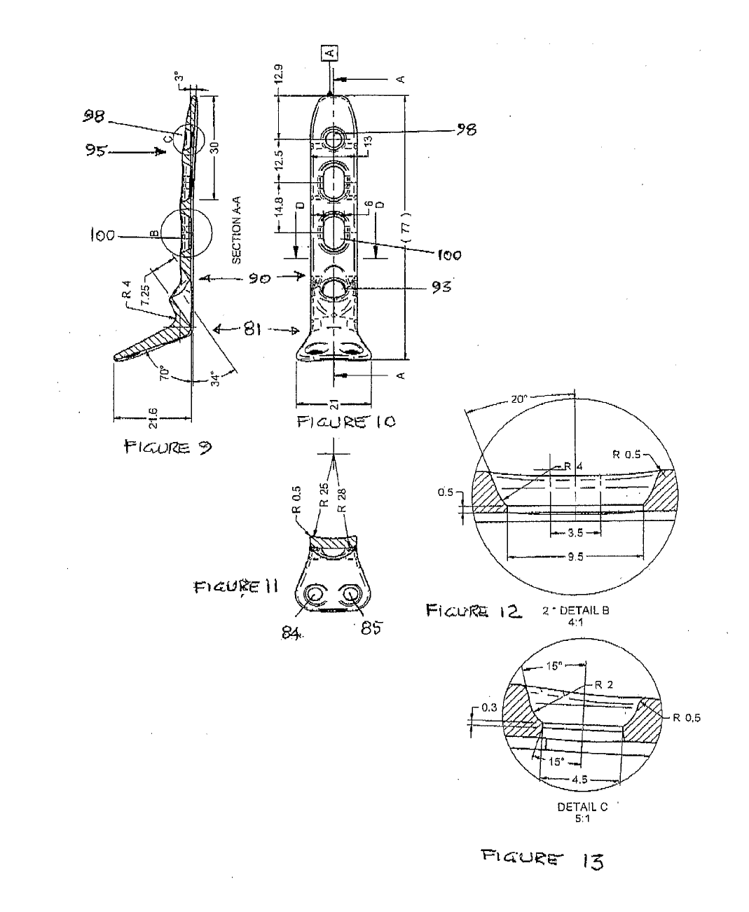

FIG. 9 shows the cross sectional side elevation view of the plate of FIG. 5 with corresponding numbering (taken along line A of FIG. 10) showing a non limiting geometry of the plate according to one embodiment. The dimensions indicated are in millimetres and indicate proportionality of the geometry of the preferred embodiment plate. Shown are distances between openings, size of openings relative angles of separate portions 81, 90 and 95 of the plate. FIG. 10 shows a front elevation view of the plate of FIG. 9 showing a non limiting geometry of the plate according to one embodiment. FIG. 11 shows a cross sectional view of the plate of FIG. 10 taken at D-D in FIG. 10 showing a non limiting geometry of the plate according to one embodiment. FIG. 12 shows an enlarged view of detail B of opening 100 in FIG. 9 showing a non limiting geometry of the opening.

FIG. 13 shows an enlarged view of detail C of opening 98 in FIG. 9 showing a non limiting geometry of the opening.

This plate significantly increases the ease of pantalar fusion and also allows the ability to start with ankle fusion and add the pantalar arthrodesis component as required. It is believed that in the pantatar fusion setting, one of the reasons for failure is necrosis of the talus, which occurs due to the multiple incisions that are needed. Avoiding a medial incision in this procedure will reduce the rate of talus necrosis after pantalar fusion. This plate also significantly increases the ease of pantalar fusion and also allows the ability to start with ankle fusion and add the pantalar arthrodesis component as required.

The specific dimensions of any of the bone fixation plate of the present invention can be readily varied depending upon the intended application, as will be apparent to those of skill in the art in view of the disclosure herein. Moreover, although the present invention has been described in terms of certain preferred embodiments, other embodiments of the invention including variations in dimensions, configuration and materials will be apparent to those of skill in the art in view of the disclosure herein. In addition, all features discussed in connection with any one embodiment herein can be readily adapted for use in other embodiments herein. The use of different terms or reference numerals for similar features in different embodiments does not imply differences other than those which may be expressly set forth. Accordingly, the present invention is not limited to the preferred embodiments disclosed herein.

The invention may also be provided as a kit including a plurality of plates and associated fixation screws. Typically a kit may comprise three to five plates for a surgeon to select from depending upon the particular anatomy of the patient. One significant advantage of the plate described herein is the oblique screw portal allowing for various angles and the ability to incorporate more joints into the arthrodesis as required. Screw sizes may be adjusted to allow for particular insertion points and specific characteristics (e.g. bone density) of bone at points of fixation. The plate may also be manufactured with varied thickness at regions requiring additional strength and contoured to a geometry to best suit ankle anatomy. Screw openings may be offset to allow three point talar fixation and plate compression. Another advantage of the plate is its pliability at regions when bending may be required for conformity with bone anatomy. The plate allows the talus to be fused with the tibia thereby providing a supporting bridge which is helpful for patients with large ankle defects. The plate also reduces the number of incisions in the patient and may be inserted through an anterior section of joint using the same incision required for a total ankle replacement. For example if a surgeon is revising a total ankle replacement, the same incision can be used as that used when removing a joint replacement and treating with arthrodesis. This techniques provides improved results in a case where large amounts of bone dissection following a total ankle replacement.

### Analysis of Simulated In Vivo Performance

Studies and testing of a preferred embodiment of the fusion plate have been conducted which determine capacity of the plate to withstand anticipated loadings. The testing considers ankle fusion plate's response to in-vivo loads. The objective of the study was to determine finite element stresses in the plate which result from applied loads and was intended to simulate as closely as possible the in vivo static and dynamic loading conditions. Mechanical properties were taken as that specified as minima in the relevant ISO standards or as specified by the material supplier.

### Equipment Used in the Testing

DELL INSPIRON 5150
ANSYS/WORKBENCH V11.0
SOLIDEDGE V 19.0

The loading regimes selected for the plate system and parameters was used for load simulation are listed below:

| Load Case | Normal Load (N) | Loaded surface |
|---|---|---|
| 1 | 1,200 | Distal face |
| 2 | 3600 | Distal face |

The plate was screw fixed along its the proximal length onto an idealised tibia. Each screw was initially rigidly fixed into the simulated bone side and had a simulated friction effect of sliding against the plate counter faces.

A second simulation was performed to determine the effects of variable bone density on the distal tibia.

Properties for the 316L stainless steel (BioDur 108) alloy were selected as Young's Modulus. E 200 GPa, Poisson's ratio=0.3 and a tensile yield strength of 938 MPa and ultimate tensile strength of 1269 MPa. The bone stiffness was varied from 0.5 GPa to 3 GPa and Poisons ratio of 0.3.

The following load conditions were simulated:

| | |
|---|---|
| 1. Screws rigidly fixed, | 1,200 N (120 kg) Distally fixed. |
| 2. Bone with low stiffness, | 1,200 N (120 kg) Distally loaded. |
| 3. Bone with high stiffness, | 1,200 N (120 kg) Distally loaded. |
| 4. Bone with low stiffness, | 3,600 N (360 kg) Distally loaded. |

The resulting stress distributions in the neck and stem were compared.

The simulations were solved as non-linear elastic stress analysis, mesh refinement was applied to the regions of high stress concentration. An initial mesh size was selected as 1 mm, the resulting stresses were recorded. A subsequent run was performed with a refined mesh around high stress locations. A mesh density of half was used and the subsequent change in stress was compared to the original run, the results of these simulations was within 10%, thus a mesh density of 1 mm was selected for all simulations,

Observations from Results.

The initial simulation (1,200 N-120 kg load) simulated the fixation screws rigidly fixed into the bone, i.e. the bone did not allow the screws to be pulled with the bone. This simulation would be analogous to hard cortical bone fixation of the screws. As would be anticipated the results showed that the highest stresses occur on the inner surface of the plate approximately were a change in section thickness occurs just above the angled screw formation. This is due to the reduction in effective (cross sectional) area of the plate at that location. The magnitude of peak tensile stress at that location was 495 MPa, which is approximately 47% less than the yield strength of the material, thus providing a factor of safety of 1.9 which well satisfies engineering design standards.

When the bone quality is reduced (stiffness of supporting bone reduces) the stress increases. The stress increases over a range from 495 to 713 to 910 MPa moving from optimally stiff bone to low bone quality respectively. The location in the plate of the high stress concentration region was unchanged with the magnitude increasing with decreased bone quality. The testing found that for poor distal tibia bone quality the factor of safety is reduced to approximately 1 as the maximum tensile stress is approximately at the yield strength of the material. The maximum deformation in the plate occurs at the tip of the distal surface (toe) of the plate and varies from 0.37 to 0.53 and 0.97 mm for the highest to lowest bone quality respectively.

A simulation was performed to determine the integrity of the plate with a 3,600N load (360 kg) on the rigidly supported “home run” lag screw. The maximum stress in the plate is in the same location as before with the exception that it has increased from 495 MPa to 1,484 MPa for the 1,200N (120 kg) and 3,600N (360 kg) loads respectively. The ultimate tensile strength of the BioDur108 alloy is approximately 1269 MPa. Thus, for the applied load of 3,600N (360 kg) the material failed at the region of the screw opening (distal tibia engagement region) where the effective cross sectional area of the plate is reduced to accommodate the fixation screw at that point.

It can be estimated that the maximum load the tested plate can take prior to failure is 3,075N (308 kg).

For perfect (rigid) tibial bone quality the plate can carry 1,200N (120 kg) with a factor of safety of 1.9. Decreasing the bone quality decreases the load carry capacity of the plate. With the lowest bone quality (stiffness of 1.0 GPa) for an applied load of 1,200N (120 kg) the stress in the plate increased to 910 MPa just below the yield strength of the material. The plate tested will only carry approximately 1,000N (300 kg) when rigidly fixed to the tibia, at a load of 3,600N (360 kg) the stress in the plate exceeds the ultimate strength of the alloy.

The above tests provide performance indications for a plate of the particular type and geometry tested. Arthrodesis plates with different geometry and dimension such as alternative thickness distributions and angulations may result in different measured loadings and plate response. Alternative plates made from different materials and different geometry will be likely to have different load capacity results for equivalent in vivo simulations but without compromise to fusion result provided the right plate is selected from the particular patient. Accordingly, the above observations and findings should be taken as a non limiting example of tested simulated in vivo performance for one plate of a particular size, geometry and material and should not be construed as limiting of load capacities and performance of alternative ankle fusion plates made in accordance with the present invention. Proportionate plate sizes may vary from patient to patient but consistency of plate performance in vivo will be geometry of the plate

It will be recognised by persons skilled in the art that numerous variations and modifications maybe made to the invention as broadly described herein without departing from the overall spirit and scope of the invention.

1. A fusion plate for arthrodesis, the plate comprising:
a plate body including a first portion disposed in a first plane and having a first bone engaging surface and a second opposing surface,
a second portion disposed in a second plane and having a first bone engaging surface and a second opposing surface,
a third portion disposed in a third plane and having a first bone engaging surface and a second opposing surface,
the first portion including at least one opening which receives at least one fixation means;
the second portion including at least one opening which receives at least one fixation means;
the third portion including at least one opening which receives at least one fixation screw.

2-16. (canceled)

17. An implant kit for arthrodesis fusion, the kit comprising:
at least one fusion plate; and
at least one set of plate fixation screws;
each plate comprising, a first portion disposed in a first plane and having inner and outer surfaces, the inner surface opposing a bone surface, the first portion including at least one opening including a formation which receives at least one bone fixation screw in a first orientation, a second portion disposed in a second plane and having inner and outer surfaces, the inner surface opposing a bone surface, the second portion including at least one opening including a formation which receives a bone fixation screw in at least one orientation, a third portion disposed in a third plane and having inner and outer surfaces, the inner surface opposing a bone surface, the third portion including at least one opening which receives at least one bone fixation screw in a first orientation.

18-54. (canceled)

55. A fusion plate for arthrodesis, the plate comprising:
a plate body including a first portion disposed in a first plane and having a first bone engaging surface and a second opposite surface,
a second portion disposed in a second plane and having a first bone engaging surface and a second opposing surface,
the first portion including at least one opening which receives at least one fixation means;
the second portion including at least one opening which receives at least one fixation means.

56-68. (canceled)
